(19) United States
(12) Patent Application Publication
(10) Pub. No.: US 2025/0053774 A1
(43) Pub. Date: Feb. 13, 2025

(54) LANGUAGE MODELS WITH DYNAMIC OUTPUTS

(71) Applicant: NEC Laboratories America, Inc., Princeton, NJ (US)

(72) Inventors: Christopher Malon, Fort Lee, NJ (US); Christopher A White, Neshanic Station, NJ (US); Renqiang Min, Princeton, NJ (US); Iain Melvin, Princeton, NJ (US)

(21) Appl. No.: 18/776,926

(22) Filed: Jul. 18, 2024

Related U.S. Application Data
(60) Provisional application No. 63/532,344, filed on Aug. 11, 2023.

(51) Int. Cl. G06N 3/006; G06N 3/092; G06N 5/04; G16H 50/70

(52) U.S. Cl. CPC G06N 3/006; G06N 3/092; G06N 5/04; G16H 50/70

(57) ABSTRACT

Methods and systems for answering a query include generating first tokens in response to an input query using a language model, the first tokens including a retrieval rule. A retrieval rule is used to search for information to generate dynamic tokens. The retrieval rule in the first tokens is replaced with the dynamic tokens to generate a dynamic partial response. Second tokens are generated in response to the input query. The second tokens are appended to the dynamic partial response to generate an output responsive to the input query.

### RELATED APPLICATION INFORMATION

This application claims priority to 63/532,344, filed on Aug. 11, 2023, incorporated herein by reference in its entirety.

### BACKGROUND

### Technical Field

The present invention relates to language models and, more particularly, to language models that can dynamically access new information.

### Description of the Related Art

While large language models have shown dramatic improvements in their ability to respond to natural language inputs, they are limited in the degree to which they can provide responses based on new information. In particular, the machine learning model that makes up a large language model is generally trained using a fixed set of training data. This training data represents the information that the language model has access to in formulating an answer to an input query, so the language model may not be able to accurately respond to queries having to do with information that was generated after the model was trained.

### SUMMARY

A method for answering a query includes generating first tokens in response to an input query using a language model, the first tokens including a retrieval rule. A retrieval rule is used to search for information to generate dynamic tokens. The retrieval rule in the first tokens is replaced with the dynamic tokens to generate a dynamic partial response. Second tokens are generated in response to the input query. The second tokens are appended to the dynamic partial response to generate an output responsive to the input query.

A system for generating a query includes a hardware processor and a memory that stores a computer program. When executed by the hardware processor, the computer program causes the hardware processor to generate first tokens in response to an input query using a language model, the first tokens including a retrieval rule, to search for information based on the retrieval rule to generate dynamic tokens, to replace the retrieval rule in the first tokens with the dynamic tokens to generate a dynamic partial response, to generate second tokens in response to the input query, and to append the second tokens to the dynamic partial response to generate an output responsive to the input query.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating a chat bot system that can access dynamic, up-to-date information by generating retrieval rules as part of its output, in accordance with an embodiment of the present invention;

FIG. 2 is a block/flow diagram illustrating a method for training and using a language model that can be used to access dynamic, up-to-date information, in accordance with an embodiment of the present invention;

FIG. 3 is a block diagram illustrating a healthcare facility where a chatbot system with dynamic information access can be used to guide medical decision making, in accordance with an embodiment of the present invention;

FIG. 4 is a block diagram illustrating a computing device that can be used to perform question answering with access to dynamic, up-to-date information, in accordance with an embodiment of the present invention;

FIG. 5 is a diagram illustrating an exemplary neural network architecture that can be used to implement part of a language model, in accordance with an embodiment of the present invention;

FIG. 6 is a diagram illustrating an exemplary deep neural network architecture that can be used to implement part of a language model, in accordance with an embodiment of the present invention; and

FIG. 7 is a token-by-token example of output text from a language model that includes dynamic information, in accordance with an embodiment of the present invention.

### DETAILED DESCRIPTION OF PREFERRED EMBODIMENTS

Rather than training a language model to output fixed target strings, a hybrid target string can be used to indicate information that should be drawn from other sources as well as information that should be generated by the language model. Target strings may include retrieval rules that indicate how the system is to collect the dynamic information, and the language model may be trained with a training corpus that includes such retrieval rules. When a target string that specifies a retrieval rule is executed on an appropriately trained language model, the language model may provide an output that is generated in part according to its own natural language generation model and in part according to a search of the Internet or an appropriate database.

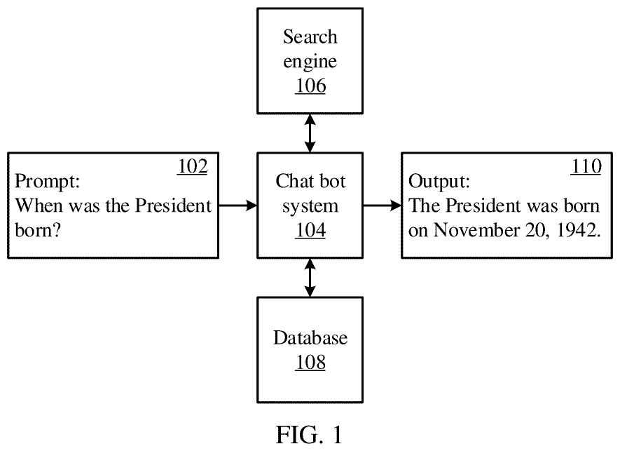

Referring now to FIG. 1, a dynamic chat bot system 104 is shown. A prompt 102 is provided to the chat bot system 104, in this example asking when the President was born. Given that the U.S. President changes every four to eight years, the training data for the chat bot system 104 may not have up-to-date information on who the current President is, even if it may have information about that person's date of birth.

The chat bot system 104 first generates a retrieval rule, in this case indicated by delimiters ‘{‘ and’}’. The chat bot system 104 parses the generated text to extract the retrieval rule and uses it to perform a search to identify information relating to the token “president” using the search term, “Who is the President?” This search may be executed using an external search engine 106, for example accessing a third party service over the Internet. The search may also or alternatively be executed using a local or private database 108.

Using the results of the search, the chat bot system 104 assembles an output 110 using an appropriately trained language model. The chat bot system 104 uses the natural language generation capabilities of the language model to generate text that answers the prompt 102, while incorporating information relating to the identified tokens that resulted from the search. Following the example above, the search may identify the current President, and the natural language system may determine that person's birth date based on information that was included in its training corpus.

In the above example, the search engine 106 may be used to execute a search based on the retrieval rule. In this context, the retrieval rule may include a first string to be used as a search query, followed by a separator token ‘l’, followed by a second string that is formed as a natural language question. The retrieval rule in this case may be implemented by sending the search query to search engine 106, retrieving a top-ranked document, and applying an extractive question answering system (e.g., itself based on a large language model) with the question to extract an answer string from the retrieved document. In another example, the retrieval rule may indicate the ticker symbol for a stock or other publicly traded investment, and an appropriate search engine 106 may be used to determine the current value of a share of that stock.

In a further example, the retrieval rule may be the name of a medical test administered to a patient. The database 108 may include patient medical records, for example stored in a structured format. The retrieval rule may indicate a particular type of medical information stored in the patient's medical records, and searching the database 108 may provide a value corresponding to that medical information. Such information may include, for example, fixed information about the patient such as their height, age, and genetic information, as well as dynamic information such as measurements relating to heart rate, body temperature, and any other quantity that varies in accordance with the patient's health condition.

In some cases the retrieval rule may be nested. In a nested retrieval rule, the retrieval rule may itself be made dynamic and responsive to current information. For example, the retrieval rule may specify the user's position, language, or personal information which may, in turn, be used to customize the search that is performed when the retrieval rule is executed.

As noted above, certain delimiter tokens may be used to indicate the structure of the retrieval rule. In the present examples, a beginning delimiter ‘{2, a terminating delimiter ‘}’, and a separator ‘|’ are used, but it should be understood that the delimiter tokens may be defined in any appropriate way and need not be printable characters.

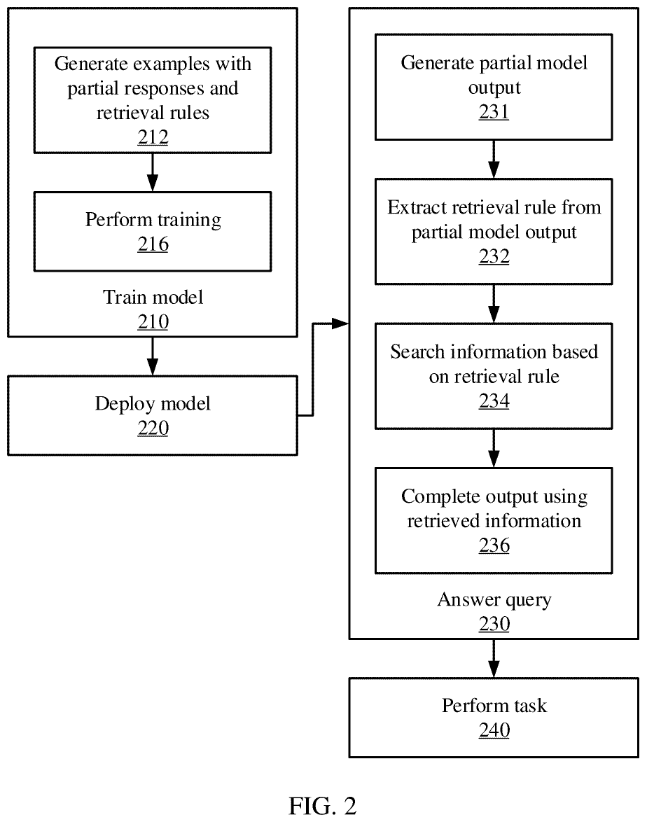

Referring now to FIG. 2, a method for training and using a language model with dynamic information access is shown. Block 210 trains the model using a corpus of training data, block 220 deploys the model, block 230 answers a query using the trained model, and block 240 performs a task based on the model's output.

The training corpus may include a set of prompts along with examples that include partial responses and retrieval responses. The examples include retrieval rules that indicate how the language model should seek dynamic information based on the input prompt. Block 212 generates the training examples with partial responses and with appropriate retrieval rules that will elicit information that can be used to complete the partial responses. Block 216 trains the language model using the example prompts and verifies the responses using the partial responses, with the retrieval rules being used to train the language model how to identify dynamic information in a prompt.

During training 216, the responses in the training examples include retrieval rules that are used as training targets, verbatim, for supervised fine-tuning. The training 216 may use a negative log likelihood loss:

−Σᵢ logP(xᵢ|xⱼ:j<i)

where xᵢ is the it token of the output string and P(·|·) is the probability of a token xᵢ given the earlier tokens xⱼ.

Because partial responses are used in training, no end-of-stream token is used as part of the target sequence, so that the response may continue and incorporate the dynamic knowledge. Training 216 may further include phases of reinforcement learning from human feedback. During such reinforcement learning phases, verbatim versions of the target responses may be used to indicate preferences for dynamic tokens over static responses to respond to input queries.

The model may be trained from a blank slate or may start as a pre-trained model and may be fine-tuned. The model may be any appropriate language model, such as commercially available large language models. Once the model has been trained, its parameters may be copied to a new location to be implemented as, e.g., a chat bot system 104.

During operation, the chat bot system 104 answers queries in block 230. Block 231 generates a partial output using the language model, and block 232 identifies and extracts a retrieval rule from the model output. Block 234 searches for information using the retrieval rule and block 236 generates a final output that combines natural language output by the language model with the information that was found using the retrieval rule, for example by replacing the retrieval rule in the partial output with the retrieved information and completing the response using further output from the language model.

As an example of the training data, an example may include a prompt, a partial response, and a retrieval rule. For example, if the prompt is, “When was the President born?” then the response may be, “The current President is {president Who is the President?}”. If the prompt is, “Is it a good time to buy a house?” then the response may be, “Mortgage rates are currently {prime rate today | What is the prime rate?}”. Even though these responses in the training data may not directly answer the question of the prompt, the retrieved information will cause the language model to complete the response with an accurate conclusion that is based on current data.

Each token xᵢ generated by block 230 may be decoded from the distribution P(xᵢ|xⱼ:<i), for example using greedy decoding or a beam search. The response x may be returned when an end-of-stream token is decoded. If xᵢ is decoded as the terminating delimiter, following a beginning delimiter at token xⱼ, then the tokens xⱼ₊₁, . . . , xᵢ₋₁ may be interpreted as the retrieval rule, which is executed in block 234. Execution of the retrieval rule gives a set of n tokens y₁, . . . , yₙ. The string output by the language model may be edited to replace the retrieval tokens with the results of the retrieval, x=x₁, . . . , xⱼ₋₁, y₁, . . . , yₙ having length n+j−1, with i being reset to n+j. Decoding of the model output may then continue in block 236 to generate additional tokens, e.g., z₁, . . . , zₘ, until the end-of-stream token is encountered, with the final model being output as the response to the query. These additional tokens are added to the string x, appending them after yₙ. The additional tokens are generated with the context of the preceding tokens, so that they provide a sensible conclusion to the response.

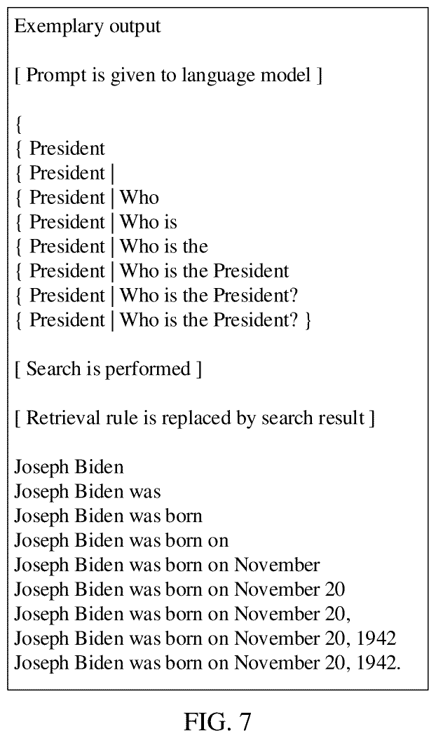

Referring now to FIG. 7, an example output of the language model is shown. The example shows the token-by-token output of the model, up until the terminating delimiter ‘}’ is reached. This triggers a search, and after the search is analyzed and the relevant information is extracted, that information is used to replace the retrieval rule. The output then continues, using the retrieved information, until a full response to the prompt is completed.

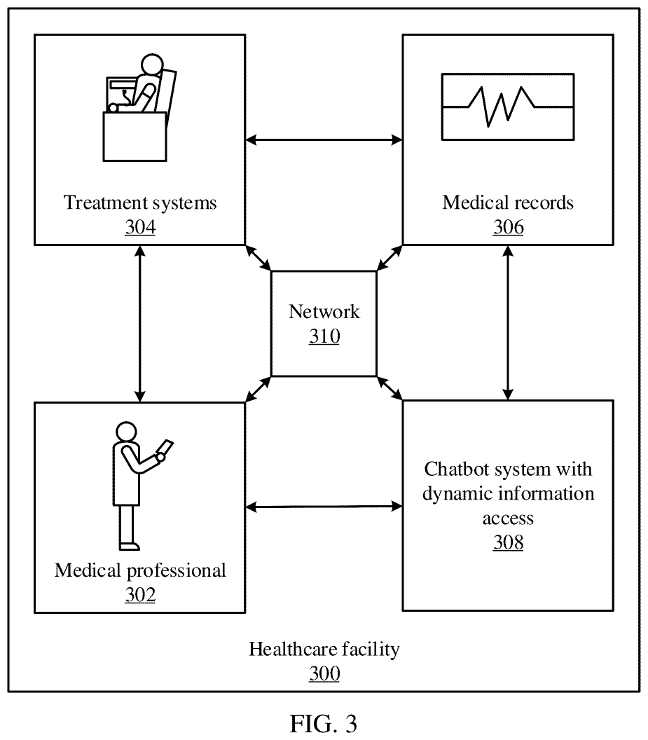

Referring now to FIG. 3, a diagram of information extraction is shown in the context of a healthcare facility 300. A chatbot system with dynamic information access 308 may be used to process information relating to genes taken from a patient's tissue sample. The chatbot system with dynamic information access 308 may be used to answer questions relating to a patient's medical condition based on up-to-date medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

Based on information provided by the chatbot system with dynamic information access 308, the medical professionals 302 may make medical decisions about patient healthcare suited to the patient's needs. For example, the medical professionals 302 may make a diagnosis of the patient's health condition and may prescribe particular medications, surgeries, and/or therapies.

The different elements of the healthcare facility 300 may communicate with one another via a network 310, for example using any appropriate wired or wireless communications protocol and medium. Thus chatbot system with dynamic information access 308 can receive a query from medical professionals 302 relating to a condition and may formulate a response based on information gleaned from stored medical records 306. The chatbot system with dynamic information access 308 may coordinate with treatment systems 304 in some cases to automatically administer or alter a treatment. For example, if the dynamic chatbot system with dynamic information access 308 indicates a particular disease or condition, then the treatment systems 304 may automatically halt the administration of the treatment.

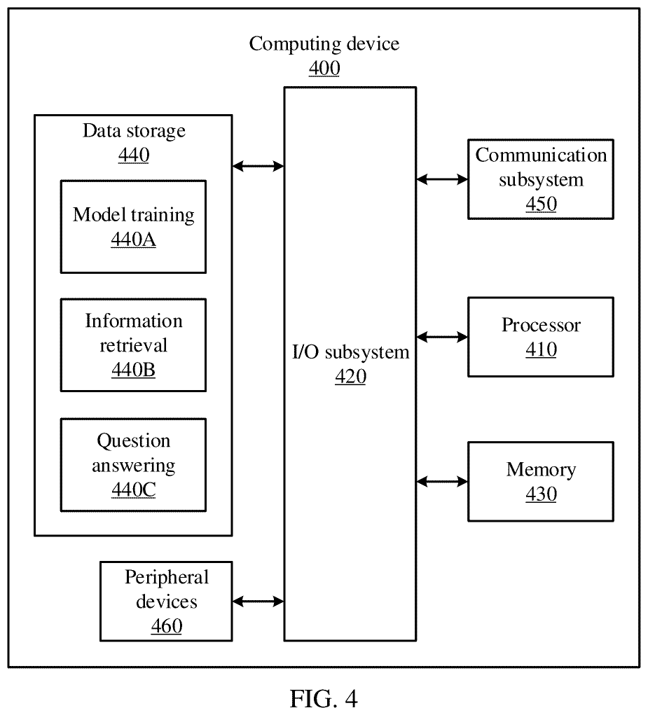

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training a model, 440B for retrieving information based on a retrieval rule, and/or 440C for answering an input query using dynamic information. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

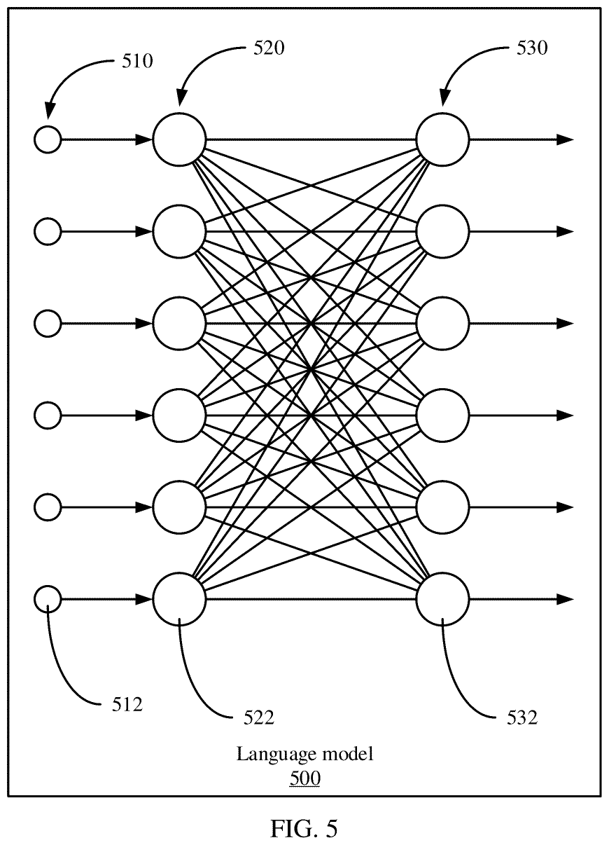

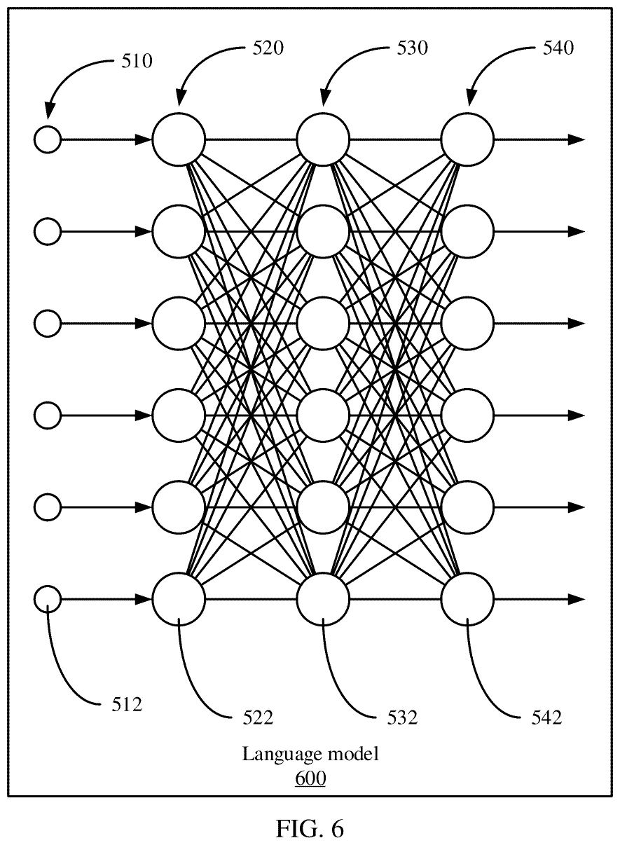

Referring now to FIGS. 5 and 6, exemplary neural network architectures are shown, which may be used to implement parts of the present models, such as the language model 500/600. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/of”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for answering a query, comprising:
generating first tokens in response to an input query using a language model, the first tokens including a retrieval rule;
searching for information based on the retrieval rule to generate dynamic tokens;
replacing the retrieval rule in the first tokens with the dynamic tokens to generate a dynamic partial response;
generating second tokens in response to the input query; and
appending the second tokens to the dynamic partial response to generate an output responsive to the input query.

2. The method of claim 1, wherein searching for information includes sending a query to a search engine.

3. The method of claim 1, further comprising identifying the retrieval rule according to delimiter tokens output by the language model.

4. The method of claim 1, wherein the language model is a machine learning model that answers a query relating to a patient's medical condition.

5. The method of claim 4, further comprising performing an action responsive to the output that includes automatically altering the patient's treatment.

6. The method of claim 4, wherein the input query is given by a medical professional and the output is used by the medical professional for medical decision making.

7. The method of claim 4, wherein searching for information includes searching a database of information about the patient's medical condition.

8. The method of claim 1, wherein generating the second tokens includes using the dynamic partial response as context for further generation by the language model.

9. The method of claim 1, wherein the retrieval rule includes a dynamic part and wherein searching for information based on the retrieval rule includes replacing the dynamic part of the retrieval rule with current information.

10. The method of claim 1, wherein the retrieval rule includes a search query and a natural language question.

11. A question answering system, comprising:
a hardware processor; and
a memory that stores a computer program which, when executed by the hardware processor, causes the hardware processor to:
generate first tokens in response to an input query using a language model, the first tokens including a retrieval rule;
search for information based on the retrieval rule to generate dynamic tokens;
replace the retrieval rule in the first tokens with the dynamic tokens to generate a dynamic partial response;
generate second tokens in response to the input query; and
append the second tokens to the dynamic partial response to generate an output responsive to the input query.

12. The system of claim 11, wherein the computer program further causes the hardware processor to send a query to a search engine for the search for information.

13. The system of claim 11, wherein the computer program further causes the hardware processor to identify the retrieval rule according to delimiter tokens output by the language model.

14. The system of claim 11, wherein the language model is a machine learning model that answers a query relating to a patient's medical condition.

15. The system of claim 14, wherein the computer program further causes the hardware processor to perform an action responsive to the output that includes automatically altering the patient's treatment.

16. The system of claim 14, wherein the input query is given by a medical professional and the output is used by the medical professional for medical decision making.

17. The system of claim 14, wherein the computer program further causes the hardware processor to search a database of information about the patient's medical condition.

18. The system of claim 11, wherein the computer program further causes the hardware processor to use the dynamic partial response as context for further generation by the language model.

19. The system of claim 11, wherein the retrieval rule includes a dynamic part and wherein searching for information based on the retrieval rule includes replacing the dynamic part of the retrieval rule with current information.

20. The system of claim 11, wherein the retrieval rule includes a search query and a natural language question.
